(19) United States
(12) Patent Application Publication
(10) Pub. No.: US 2004/0143224 A1
(43) Pub. Date: Jul. 22, 2004

(54) Method and apparatus for inhibiting fluid loss from a syringe

(76) Inventors: Jeffrey Field, Camarillo, CA (US); John Dunec, Palo Alto, CA (US)

Correspondence Address: John D McConaghy, Fulbright & Jaworski, Twenty Ninth Floor, 865 South Figueroa, Los Angeles, CA, 90017-2571, US

(21) Appl. No.: 10/450,573

(22) PCT Filed: Jan. 7, 2002
(86) PCT No.: PCT/US02/00469

(51) Int. Cl. A61M 5/32

(52) U.S. Cl. 604/195

(57) ABSTRACT

The present invention is directed to syringes that have needles that retract after an injection to provide safe syringes which reduce the possibility of a sharps injury through retraction of the needle after use into the barrel of the syringe. The syringe includes a barrel, a needle assembly positioned in the hollow body, a fluid retention system positioned in the hollow body, and a plunger assembly that moves in the hollow body. A fluid retention system retains in the syringe any fluid remaining in the needle after the plunger assembly approaches tha distal end of the hollow body. The fluid retention system includes a variable volume vacuum cavity in fluid communication with the needle passage, the vaccum cavity expanding prior to retraction of the needle assembly and providing a pressure drop in the needle, tending to draw into the hollow body any fluid remaining in the needle.

Under 35 U.S.C. 119(e), applicant claims the benefits of and priority based on provisional patent application serial No. 60/260,096, filed Jan. 5, 2001 and entitled “Syringe That Inhibits Fluid Loss Injections”, and provisional patent application serial No. 60/264,554, filed Jan. 26, 2001 and entitled “Syringe That Inhibits Fluid Loss After Injection,” the disclosures of which are hereby incorporated by reference as if fully disclosed herein.

### BACKGROUND OF THE INVENTION

The field of the present invention relates generally to apparatus and methods for protection against an accidental sharps injury or stick from an unprotected needle and for inhibition of fluid loss from a syringe.

For some time, the art has recognized the desirability of protecting personnel from accidental sharps injuries or needle sticks and against contact with fluid that might leak, drip, or be sprayed from a syringe after the syringe is used to deliver an injection. Oftentimes, after a syringe is used to inject fluid into a patient, some fluid remains in the syringe, particularly at the tip of the needle. This fluid may include the fluid injected into the patient from the syringe, and may also include bodily fluids from the patient, such as blood. Any fluids remaining in the syringe after use of the syringe may leave the syringe, such as by leaking, spraying or dripping from the syringe, and may contact persons or objects in the area. Syringes with retractable needles may be especially prone to this loss of fluid when the needle quickly retracts into the barrel of the syringe after injection.

More recently, concerns have been expressed about the possibility of transmitting serious or potentially fatal infection as a result of such accidents. Most recently, legislation requiring the use of safe needle technology is pending in a number of States and before the Occupation Safety and Health Administration. Although the art has recognized the desirability of protecting against accidental sharps injuries or needle sticks, it is believed that available devices allow fluid spraying or other loss from a syringe after injection, or require the syringe user to carry out further or non-routine steps in addition to those required to use a conventional syringe to inhibit fluid loss. The further a device is from routine operation, the less accepted it is by the medical community.

Various retractable syringes are disclosed in co-owned PCT Application No. PCT/US97/20646, International Publication No. WO 98/20923, the disclosure of which is hereby incorporated by reference as if fully set forth herein. While these devices operate successfully, it has been found that the devices may be improved by inhibiting fluid loss after injection.

Another concern with prior art devices is the complicated and costly manufacturing processes. With the tremendous number of syringes and other needle devices used by the medical community, any substantial rise in cost of the products is undesirable and generally unacceptable.

Accordingly, there is a need for syringes, both retractable and non-retractable, that inhibit or prevent fluid loss after the syringe is used and that can be used in a conventional manner and do not require elaborate manufacturing.

### SUMMARY OF THE INVENTION

The present invention is directed to syringes that have needles that retract after an injection. Various configurations are contemplated, presented as preferred embodiments. A principle object of the present invention is to provide safe syringes which reduce the possibility of a sharps injury through retraction of the needle after use into the barrel of the syringe. Other and further objects and advantages will appear hereinafter

The syringe includes a barrel, a needle assembly positioned in the hollow body, a fluid retention system positioned in the hollow body, and a plunger assembly that moves in the hollow body. In some embodiments, the needle assembly is retractable.

In a first separate aspect of the present invention, a fluid retention system retains in the syringe any fluid remaining in the needle after the plunger assembly approaches the distal end of the hollow body.

In a second separate aspect of the present invention, the fluid retention system includes a variable volume vacuum cavity in fluid communication with the needle passage, the vacuum cavity expanding prior to retraction of the needle assembly and providing a pressure drop in the needle, tending to draw into the hollow body any fluid remaining in the needle.

In a third separate aspect of the present invention, the fluid retention system includes a variable volume vacuum cavity that, during initial retraction of the mandrel, is substantially sealed against fluid communication other than to provide fluid communication with the needle.

In a fourth separate aspect of the present invention, the fluid retention system includes a cylinder defined within the needle assembly and a piston connected with the mandrel and slidingly and sealingly engaging the cylinder during initial retraction of the mandrel.

In a fifth separate aspect of the present invention, a method of inhibiting fluid loss from a syringe following injection includes the step of reducing pressure in the needle passage after the plunger assembly has been substantially completely depressed.

In a sixth separate aspect of the present invention, it is contemplated that combinations of the foregoing separate aspects may be incorporated into a single embodiment.

### BRIEF DESCRIPTION OF THE DRAWINGS

FIG. 1 is an expanded view of a retractable syringe in accordance with the present invention.

FIG. 2 is a cross sectional view of one embodiment of the syringe barrel.

FIG. 3 is an exploded view of the closed end of the syringe barrel of FIG. 2.

FIG. 4 is an exploded view of another embodiment of the closed end of the syringe barrel.

FIG. 5 is a side elevation of one embodiment of the needle assembly.

FIG. 6 is a side elevation of another embodiment of the needle assembly.

FIG. 7 is an exploded view of the closed end of another embodiment of the syringe barrel and another embodiment of the needle assembly.

FIG. 8 is an isometric view of one embodiment of a plunger assembly.

FIG. 9 is a cross sectional view of the plunger frame of the plunger assembly of FIG. 8.

FIG. 10 is a cross sectional view of another embodiment of a plunger assembly.

FIG. 11 is an enlarged view of part of the plunger assembly of FIG. 10.

FIG. 12 is one embodiment of a retraction assembly.

FIG. 13 is one embodiment of a catch member.

FIG. 14 is an exploded view of the sealing end of the plunger frame of FIG. 9.

FIG. 15 is a cross sectional view of another embodiment of the sealing end of a plunger frame.

FIG. 16 is a cross sectional view of the plunger assembly of FIG. 8.

FIG. 17 is an exploded view of the first end of the plunger assembly of FIG. 16.

FIG. 18 is a side elevation view of one embodiment of the mandrel of the present invention.

FIG. 19 is a side elevation view of another embodiment of the mandrel and a cross sectional view of the mandrel seal of the present invention.

FIG. 20 is a side elevation view of one embodiment of the catch member of the present invention.

FIG. 21 illustrates an embodiment of the syringe in an assembled but unused condition.

FIG. 22 illustrates the syringe of FIG. 21 upon initial substantial depression of the plunger assembly.

FIG. 23 illustrates the syringe of FIG. 21 after loading of the syringe.

FIG. 24 illustrates the forward portion of the syringe of FIG. 21 as it is inserted in a patient.

FIG. 25 illustrates the forward portion of the syringe of FIG. 21 upon substantial injection depression of the plunger assembly.

FIG. 26 illustrates the forward portion of the syringe of FIG. 21 after the mandrel tip has entered the needle assembly cavity.

FIG. 27 illustrates the forward portion of the syringe of FIG. 21 upon complete depression of the plunger assembly.

FIG. 28 illustrates the syringe of FIG. 21 after retraction of the needle.

FIG. 29A is a side elevation of a first embodiment of a fluid retention system.

FIGS. 29B and 29C are a cross-sectional view of the fluid retention system of FIG. 29A.

FIG. 30 is a cross-sectional view of a second embodiment of a fluid retention system.

FIG. 31 is a side elevation of a third embodiment of a fluid retention system.

FIG. 31A is a top view of the fluid retention system of FIG. 31.

FIGS. 32A and 32B are cross-sectional views of a fourth embodiment of a fluid retention system.

FIG. 33A is a cross-sectional view of a sixth embodiment of a fluid retention system.

FIG. 33B is a perspective view of the mandrel of the fluid retention system of FIG. 33A.

FIG. 34A is a cross-sectional view of a seventh embodiment of a fluid retention system.

FIG. 34B is a perspective view of the washer of the fluid retention system of FIG. 34A.

FIG. 35A is a cross-sectional view of an eighth embodiment of a fluid retention system.

FIG. 35B is a perspective view of the fluid retention system of FIG. 35A.

### DETAILED DESCRIPTION OF THE PREFERRED EMBODIMENTS

The preferred embodiments will be described with reference to drawing figures.

A. Retractable Syringes

Various retractable syringes are disclosed in co-owned PCT Application No. PCT/US97/20646, International Publication No. WO 98/20923, the disclosure of which is hereby incorporated by reference as if fully set forth herein.

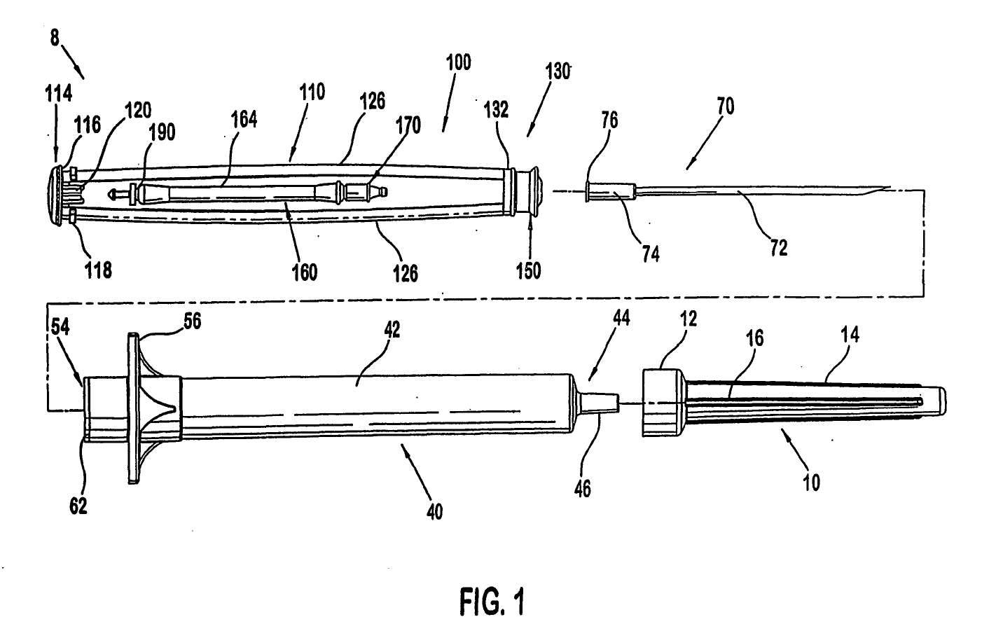

Referring to FIG. 1, an embodiment of a syringe assembly 8 is comprised generally of a cap member 10, a syringe barrel 40, a needle assembly 70, and a plunger assembly 100.

The cap member 10 includes an open, mating end 12 and a closed cone section 14. The mating end 12 is preferably configured to slidingly engage the syringe barrel 40. Alternatively, the mating end 12 may be provided with threads (not shown) which may engage corresponding threads (not shown) on the syringe barrel 40. Other cap and corresponding barrel configurations are known and may also be employed. The closed cone section 14 preferably includes a plurality of ribs 16 which assist gripping of the cap member 10.

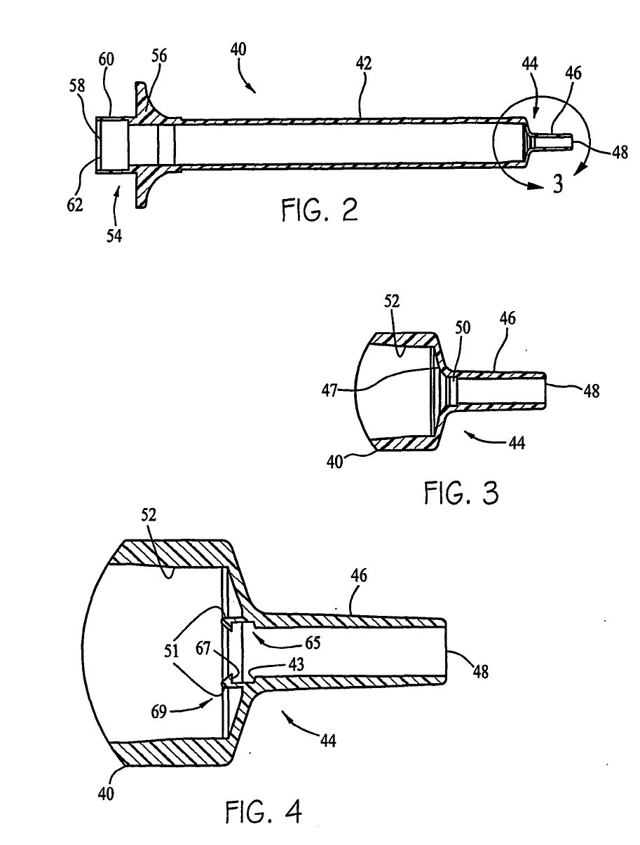

Referring to FIGS. 2 and 3, one embodiment of the syringe barrel 40 is comprised of a hollow body portion 42 which has a closed end 44 and an open end 54. An external stabilized grip member 56 extends from the body 42 adjacent to, but forward of the open end 54. The grip member 56 may have various configurations, the preferred elliptical configuration being shown. An internal annular shoulder 60 is defined in the hollow body 42 at approximately the same position as the grip member 56. The open end 54 defines an open cavity 58 rear of the internal annular shoulder 60. An internal annular lip 62 may also be provided adjacent the open end 54.

The closed end 44 is defined by a truncated cone 46 which includes a truncating plane having an aperture 48. Referring to FIG. 3, in one embodiment of the syringe barrel 40, a retaining groove 50 is located on the interior of the syringe barrel 40 at a position adjacent to the closed end 44. The retaining groove 50 retains the needle assembly 70 in position during use as will be described in more detail hereinafter. In one embodiment, the closed end 44 proximate the truncated cone 46 has a generally convex taper 47 and at least one internal ramp 52, the functions of which will be described hereinafter.

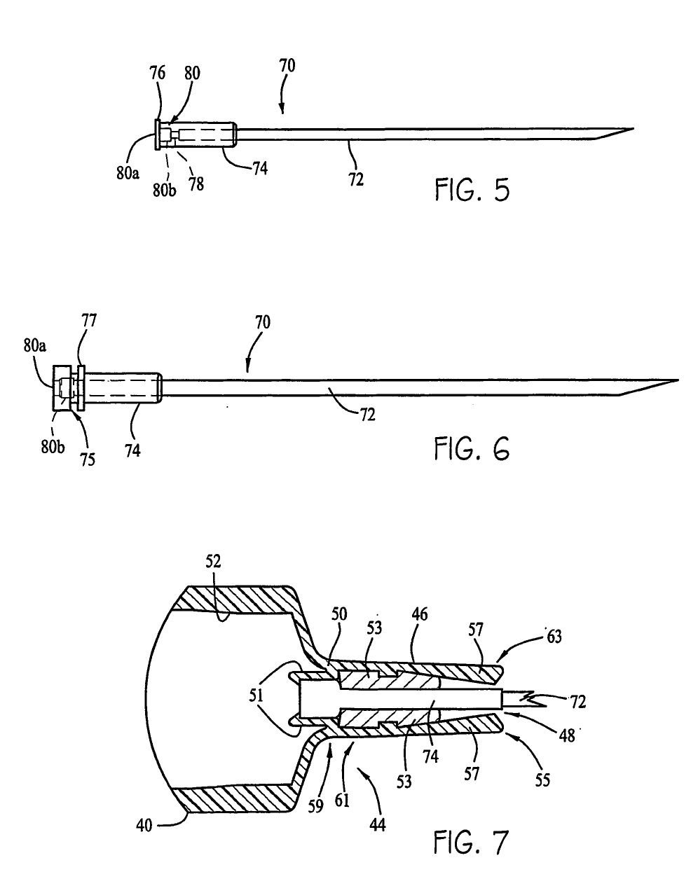

Referring to FIG. 5, in another embodiment of the syringe barrel 40, retaining fingers 51 are attached to the interior of the syringe barrel 40 at a position adjacent to the closed end 44. The retaining fingers 51 retain the needle assembly 70 in position prior to retraction of the needle as will be described in more detail hereinafter.

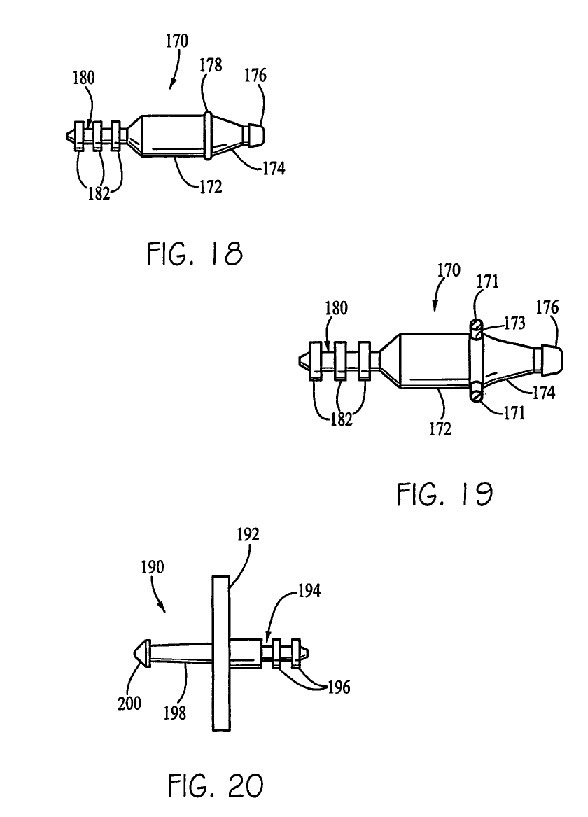

FIG. 5 depicts one embodiment of the needle assembly 70. In this embodiment, the needle assembly 70 is comprised of a needle 72 which is centrally positioned in the hollow projection 74. The hollow projection 74 generally complements the interior of the truncated cone 46 of the syringe barrel 40. Immediately adjacent to the projection 74 is a sealing ring 76. The projection 74 and the sealing ring 76 preferably are formed as a unitary molding, but may be formed as separate components. The interior passage 78 of the needle assembly 70 is in fluid communication with the hollow needle 72 and the geometrically configured cavity 80 extending into the rear surface of the needle assembly 70. The cavity 80 preferably has a cylinder portion 80a and a hemispheric portion 80b which complement the geometrically configured tip 176 of the plunger mandrel 170 (shown in FIG. 18). The needle assembly 70 is positioned within the syringe barrel 40 such that the needle 72 extends through the aperture 48 and the sealing ring 76 is positioned in and retained by the retaining groove 50. Preferably, the sealing ring 76 sealingly engages the truncated cone 46 of the syringe barrel 40 and the hollow projection 74.

FIG. 6 depicts another embodiment of the needle assembly 70. In this embodiment, the needle assembly 70 has a needle seal lip 75 that engages and retains a needle seal 77. The needle seal lip 75 may comprise an annular collar or other surface that retains the needle seal 77. When the needle assembly 70 is inserted into the truncated cone 46 of the syringe barrel 40, the needle seal 77 is positioned between and sealingly engages the needle seal lip 75 and the closed end 44 of the syringe barrel 40. Alternately, the needle seal 77 may sealingly engage the hollow projection 74 and the closed end 44 of the syringe barrel 40. To facilitate sealing engagement of the needle seal 77 with the closed end 44 of the syringe barrel 40, the closed end 44 may have a shelf 43 (shown in FIG. 4) that engages the needle seal 77. The needle seal 77 is preferably annular, and may comprise an O-ring.

Other embodiments of the syringe barrel 40 and needle assembly are shown in FIG. 7. In these embodiments, a needle seal 53 (shown in cross-section) is inserted through the aperture 48 at the closed end 44 of the syringe barrel 40. The needle seal 53 is positioned between and sealingly engages the hollow projection 74 and the truncated cone 46. In this embodiment, a smaller end 55 of the truncated cone 46 opens up to receive the needle seal 53 and then closes to retain the seal 53 in sealing engagement. To accomplish this opening and closing, the smaller end 55 of the truncated cone 46 is comprised of a plurality of retaining arms 57. A first end 61 of each retaining arm 57 is attached to a larger end 59 of the truncated cone 46. The first ends 61 are arranged about the circumference of the larger end 59. Each of the retaining arms 57 has a second end 63 that may move between an open position and a closed position when the retaining arms 57 are bent. An area defined by the second ends 63 of the retaining arms 57 while in the closed position is smaller than an area defined by the second ends 63 of the retaining arms 57 while in the open position. The area defined by the arms 57 in the closed position is smaller than a cross-sectional area of the needle seal 53 so that the needle seal 53 will not pass out through the aperture. When the needle seal retainer is in the closed position, the needle seal 53 sealingly engages the truncated cone 46 and the hollow projection 74, and when the needle seal retainer is in the open position, the needle seal 53 is preferably released from sealing engagement with the truncated cone 46 and the hollow projection 74. Retaining fingers 51 are attached to the interior of the syringe barrel 40 at a position adjacent to the closed end 44. The retaining fingers 51 help retain the needle assembly 70 in position prior to retraction of the needle as will be described in more detail hereinafter.

In the embodiments described herein, the needle assembly 70 is retained in a position that is adjacent to the closed end 44 of the barrel 40 by a needle assembly retainer prior to retraction. In the embodiments shown in FIGS. 3 and 5, the needle assembly retainer comprises the sealing ring 76 that is positioned in and retained by the retaining groove 50. In the embodiment shown in FIGS. 4 and 7, the needle assembly retainer comprises the retaining fingers 51.

The retaining fingers 51 are preferably disposed about the circumference of a larger end 59 of the truncated cone 46. A first end 65 of each of the retaining fingers 51 is connected with the syringe barrel 40 at a location adjacent to the closed end 44. The retaining fingers 51 preferably comprise L-shaped members with a retaining lip 67 near a second end 69. The retaining fingers 51 are bendable between a closed position and an open position. While in the closed position, the second ends 69 of the fingers 51 define an area that is smaller than the area defined by the second ends 69 when they are in the open position. The smaller area defined by the fingers 51 in the closed position is smaller than a cross-sectional area of the needle assembly 70, such that the needle assembly does not pass through the needle assembly retainer when the fingers 51 are in the closed position.

Referring to FIGS. 1 and 8-20, the plunger assembly 100 includes a plunger frame 110, a retraction assembly 160, a thumb pad 104 and a sealing member 150. The plunger frame 110 includes a first end 114 and a sealing end 130 with a pair of opposed connecting rods 126 extending therebetween. The opposed connecting rods 126 define opposed retraction assembly guide tracks 128.

As shown in FIG. 9, in one embodiment of the plunger frame, the first end 114 of the plunger frame 110 includes a terminating plate 116 extending between and bridging the opposed connecting rods 126. An annular thumb pad retaining ring 117 extends about the terminating plate 116. Additionally, a guide member 118 may extend outward from each connecting rod 126 proximate the terminating plate 116. A retention assembly 120 extends inward from the terminating plate 116 between the opposed connecting rods 126. This embodiment of the retention assembly 120 includes a pair of opposed L-shaped members 122, each L-shaped member having a beveled catch 124 extending therefrom. Other retention assemblies which permit inward passage and then retention of a geometrically configured tip are within the scope of the invention.

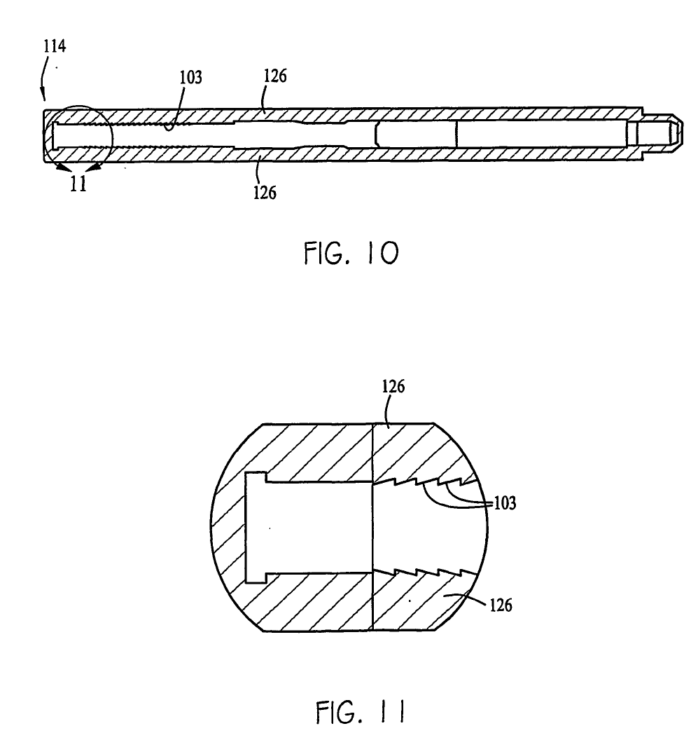

As shown in FIGS. 10 and 11, another embodiment of the plunger frame has a retention assembly that comprises a plurality of retention teeth 103 arranged along a surface of the connecting rods 126 of the plunger assembly, substantially parallel to a longitudinal axis of the elongated frame portion of the plunger assembly.

Each component of the plunger frame 110 is preferably manufactured from polypropylene or glass filled polypropylene. Other materials, including various plastics, may also be used. As described in more detail hereinafter, the plunger frame 110, in addition to components of the retraction assembly 160, is preferably formed as a first shot of a multiple shot injection molding procedure.

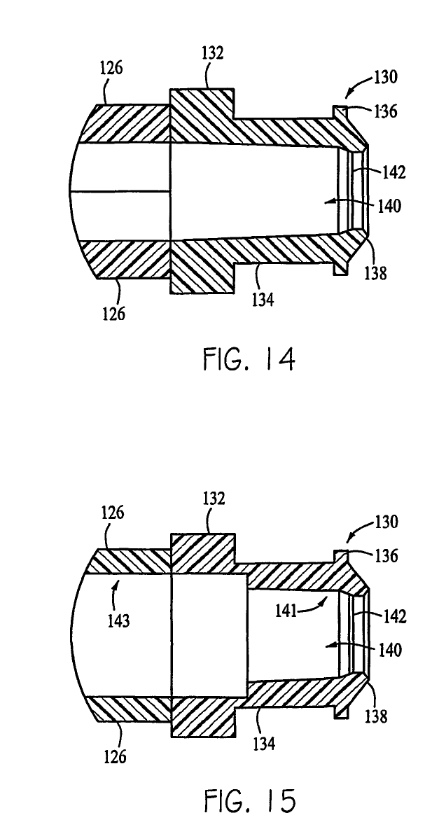

As shown in FIG. 14, in one embodiment of the plunger assembly, the sealing end 130 includes a sealing platform 132 extending between the connecting rods 126 and including an apertured cylinder 134 terminating in an apertured pressure cone 138. The apertures are preferably concentric such that a continuous hollow integral shaft 140 passes through the sealing end 130 from the sealing platform 132 to the pressure cone 138. The hollow shaft 140 is preferably tapered such that the diameter is greater within the sealing platform 132 than within the pressure cone 138. Additionally, an internal annular ring 142 extends into the hollow shaft 140 proximate the pressure cone 138. An external annular retaining ring 136 is positioned about the juncture of the cylinder 134 and pressure cone 138. The function of the taper aid the internal and external rings 136 and 142 will be described in more detail hereinafter.

As shown in FIG. 15, in another embodiment of the plunger assembly, the hollow shaft 140 has a first end 141 and a second end 143, wherein the first end 141 defines a cross-sectional area that is smaller than a cross-sectional area defined by the second end 143. The transition between the smaller and larger cross-sectional areas is depicted to be abrupt, but may alternately be gradual.

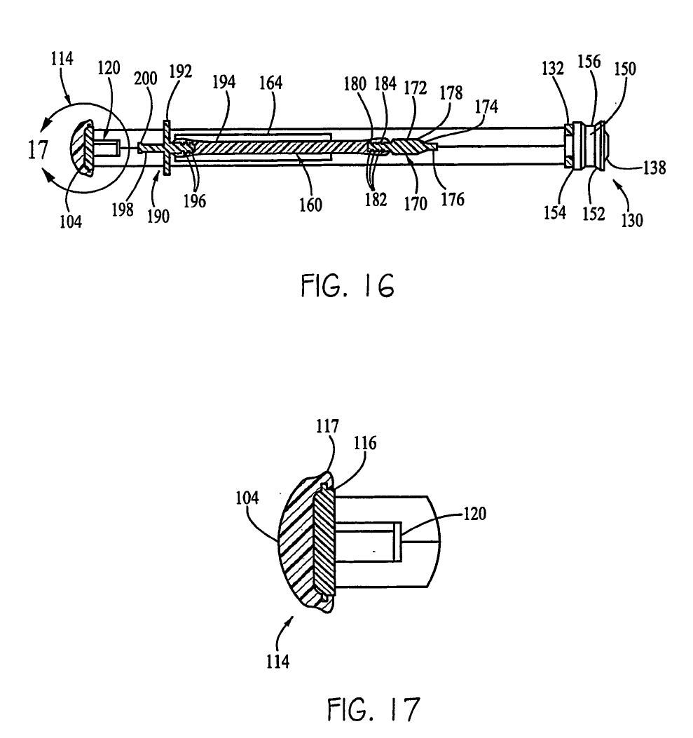

Referring to FIG. 16, the plunger sealing member 150 is positioned about the cylinder 134 and the external retaining ring 136 and is maintained in position by the external ring 136. The sealing member 150 includes annular seals 152 and 154 at each end with a narrower portion 156 positioned therebetween. When the plunger assembly 100 is positioned in the syringe barrel 40, annular seal 152 sealingly engages the inside surface of the hollow body portion 42 with an area of open space about the narrower portion 156. Annular seal 154 may also sealingly engage the hollow body 142, but may also include a passage to prevent creating a vacuum in the narrower portion 156. The sealing member 150 is preferably manufactured from an elastomer. A material found to be suitable is Kraton™ manufactured by Shell Oil. A preferred material is Kraton™ G2706 manufactured by Shell Oil. As will be described in greater detail hereinafter, the sealing member 150 is preferably overmolded directly in position, but may be manufactured separately and subsequently positioned about the cylinder 134 and retaining ring 136.

The first end 114 of plunger frame 110 can be utilized with the terminating plate 116 and no thumb pad 104. However, it is preferable to provide a thumb pad 104 about the terminating plate 116 and retained by the retaining ring 117 as shown in FIG. 17. The thumb pad 104 is also preferably manufactured from an elastomer, preferably Kraton™. As with the sealing member 150, it is preferable that the thumb pad 104 be overmolded directly in position, but it too may be manufactured separately and subsequently positioned and secured about the terminating plate 116.

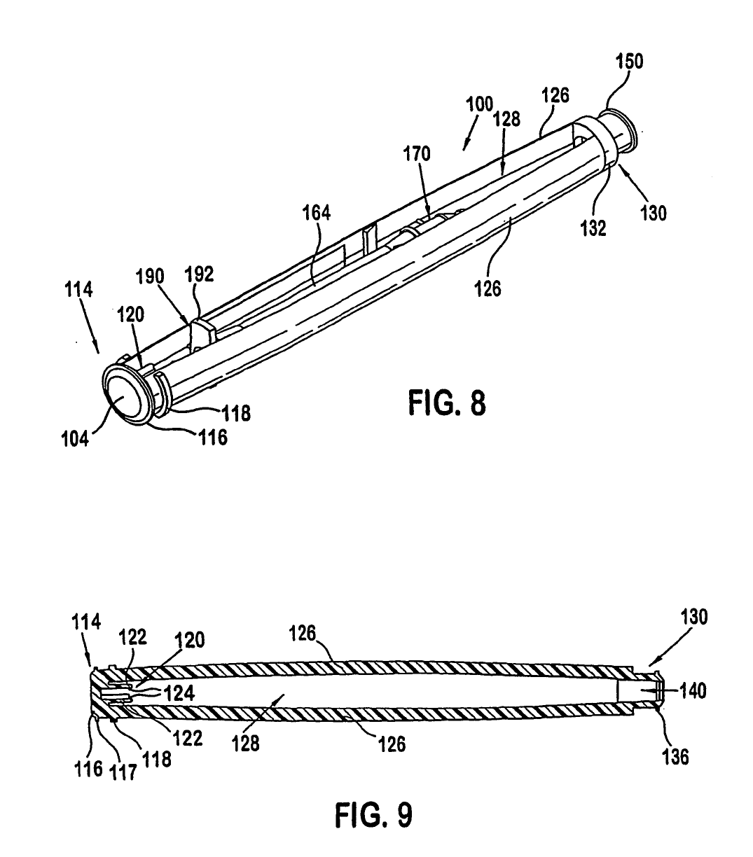

The preferred retraction assembly 160 will be described with reference to FIGS. 8, 12, 13, 16, 18, 19 and 20. The retraction assembly 160 includes a mandrel 170, a catch member 190 and an elastic member 164.

Referring to FIG. 18, one embodiment of the mandrel 170 includes a generally cylindrical body 172 with a tapered portion 174 extending from one end and a shaft portion 180 extending from the other. The tapered portion 174 terminates in a geometrically configured tip 176. A mandrel annular retaining ring 178 extends about the cylindrical body 172 proximate the juncture with the tapered portion 174. The mandrel 170 is releasably secured to the plunger frame by a retention means, which includes the retaining ring 178. The mandrel retaining ring 178 preferably is part of the mandrel 170 and does not separate from the mandrel 170. The shaft portion 180 includes a plurality of barbs 182 or the like extending therefrom for retaining the elastic member 164.

In another embodiment of the mandrel 170, as shown in FIG. 19, the cylindrical body 172 has a groove 173 and a mandrel seal 171 (a cross-section of which is depicted) is positioned about the groove 173 between the mandrel 170 and the hollow shaft 140. The mandrel seal 171 releasably and sealingly engages the mandrel 170 and the hollow shaft 140. The mandrel seal 171 is depicted as an O-ring, but may be of any configuration that releasably and sealingly engages the hollow shaft 140 and the mandrel 170.

The mandrel seal 171 initially is in sealing engagement with the mandrel 170 and the first end 141 of the hollow shaft 140 and inhibits the passage of fluid therebetween prior to retraction of the needle. The sealing engagement creates holding forces between the hollow shaft 140 and the mandrel seal 171, as well as between the mandrel seal 171 and the mandrel 170. The holding forces inhibit movement of the seal 171 relative to the mandrel 170 and relative to the hollow shaft 140, and thus inhibit retraction of the needle. Accordingly, the retention means that releasably secures the mandrel 170 to the plunger frame includes the mandrel seal 171.

In the smaller-diameter area, the holding force between the mandrel seal 171 and the mandrel 170 is greater than the holding force between the mandrel seal 171 and the hollow shaft 140, such that the mandrel seal 171 does not move relative to the mandrel 170, but instead moves relative to the hollow shaft 140 when the mandrel 170 is moved relative to the hollow shaft 140.

The desired distribution of holding forces may provided by various means. Preferably, the mandrel 170 has an annular groove 173 about the circumference of the mandrel 170 and located at a seal position on the mandrel 170. The annular groove 173 is configured so that the mandrel seal 171 abuts or fits into the groove 173 and is held in place on the mandrel 170 at the seal position so long as the mandrel seal 171 is located in the smaller-area portion of the hollow shaft 140. Once the mandrel seal 171 moves from the smaller area to the larger area of the hollow shaft 140, the mandrel seal 171 may either remain in the groove 173 or expand away from the groove 173, as described in greater detail below.

It is noted that configurations other than a groove may also be employed to retain the mandrel seal fixed with respect to the mandrel. For example, the mandrel seal could be positioned between two annular collars on the mandrel. Additionally, any other non-smooth or irregular surface may be employed to mechanically inhibit movement of the seal relative to the mandrel. Alternately, an adhesive could be used to attach the mandrel seal to the mandrel. The mandrel seal could also be formed as an integral part of the mandrel.

Referring to FIG. 16, in one embodiment of the retraction assembly 160, an annular stop 184 extends about the cylindrical body 172 of the mandrel 170 adjacent the end of the hollow shaft. The stop 184 is preferably elastomeric and therefore is preferably formed in conjunction with the elastic member 164.

Referring to FIGS. 8 and 20, one embodiment of the catch member 190 includes an elongated plate 192 which is sized such that each end of the elongated plate 192 extends into and travels within a respective retraction assembly guide track 128 of the plunger frame 110. Extending from one side of the elongated plate 192 is a shaft 194 with barbs 196 or the like extending therefrom for retaining the elastic member 164. In the embodiment shown in FIG. 20, a second shaft 198 extends from the opposite side of the elongated plate 192 and terminates in a geometrically configured catch tip 200. The tip 200 is configured to mate with and be retained by the retention assembly 120 of the plunger frame 110.

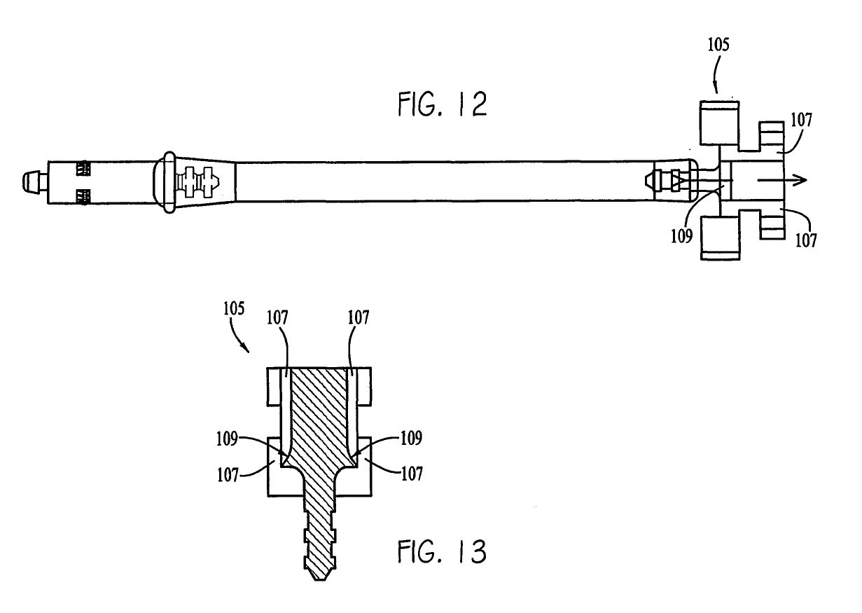

As shown in FIGS. 12 and 13, another embodiment of the catch member 105 has a guide 107 configured to travel in the guide track 128 between the connecting rods of the plunger frame 110. The catch member 105 has a catch tooth 109 situated on either side of the catch member 105. Each catch tooth 109 engages with one of the retention teeth 103 (the retention teeth 103 are part of the retention assembly shown in FIGS. 10 and 11) at a time. After one of the retention teeth 103 receives one of the catch teeth 109, the catch tooth 109 is inhibited from moving away from the first end 114 of the elongated frame portion. Accordingly, the catch member 105 is retained by the retention teeth 103 of the retention assembly and the elastic member 164 is thereby held in tension on one end by the sealing platform and on the other end by the catch member 105.

The mandrel 170 and the catch member 190 105 are preferably manufactured from the same material as the plunger frame 110. As such, these components can also be formed during the first shot of the multiple shot injection molding procedure used to form the plunger frame 110.

The elastic member 164 extends between the mandrel 170 and the catch member 190 105. The elastic member 164 is manufactured from a resilient material, which is preferably an elastomer, but which can be other materials, for example a stainless steel spring or the like. The elastic member 164 is preferably manufactured from Kraton™. In the preferred method of manufacture described below, the elastic member 164 is formed between the mandrel 170 and the catch member 190 105 with a second injection, overmolding shot. Do to the elastic member 164 being directly overmolded over the barbed shafts 180 and 194 and the innate bonding property of the preferred material, there is generally not a need for additional securing means, for example adhesive, to maintain the elastic member 164 secured to the mandrel 170 and catch member 190 105. It is contemplated that the elastic member 164, irrespective of the material from which it is manufactured, may also be manufactured separately and secured to the mandrel 170 and catch member 190 105. Since the sealing member 150, thumb pad 104 and stop 184 are all also preferably manufactured from the same material as the elastic member 164, they are also preferably formed during the second injection, overmolding shot.

Having described the components of the preferred retractable syringe 8, its assembly and use will now be described with reference to FIGS. 21-28. The needle assembly 70 is positioned in the syringe barrel 40 with the needle 72 extending through the aperture 48.

In the embodiment shown in FIGS. 3 and 5, the needle assembly 70 is inserted until the sealing ring 76 seats in and is retained by the syringe body retaining groove 50. In the embodiment shown in FIGS. 4 and 6, the needle assembly 70 with the needle seal 77 is inserted into the truncated cone 46 of the syringe barrel 40 and is retained by the retaining fingers 51. When the needle assembly 70 is being inserted into the truncated cone 46, it contacts the retaining fingers 51. Further insertion of the needle assembly 70 moves the retaining fingers 51 into the open position so that the needle assembly 70 may pass through the opening defined by the retaining fingers 51.

When the needle assembly 70 is inserted sufficiently into the truncated cone 46, the retaining fingers 51 return to the closed position. The retaining fingers 51 are formed from an elastic material so that they return to the closed position if they are displaced from the closed position and no external forces are acting on the retaining fingers 51. If a force is applied tending to push the needle assembly 70 out of the truncated cone 46 toward the open end of the barrel 40 when the retaining fingers 51 are in the closed position, the needle assembly 70 will contact at least one of the retaining lips 67, and at least one of the retaining fingers 51 will resist that force. The orientation of the surface of the lips 67 in a substantially perpendicular relationship to the direction in which such a force would be applied decreases the component of the force that would act to spread the fingers 51 into the open position.

In the embodiment shown in FIG. 7, the needle seal 53 is inserted into the truncated cone 46 through the aperture 48 rather than through the open end of the syringe barrel 40. To insert the needle seal 53 into the truncated cone 46 through the aperture 48, the retaining arms 57 are moved into the open position and held in the open position so that the needle seal 53 may pass through the opening defined by the arms 57. Once the needle seal 53 is sufficiently inserted into the truncated cone 46, the arms 57 are released and move toward the closed position. The arms 57 are formed of an elastic material so that the arms 57 return to the closed position if they are displaced from the closed position and no external forces are acting on the arms 57. Once the needle seal 53 is inserted into the truncated cone 46 and the arms 57 return to the closed position, the needle seal 53 is retained in the truncated cone 46 by the arms 57.

The needle assembly 70 may be inserted through the aperture 48 or through the open end of the barrel 40. If the needle assembly 70 is inserted through the open end of the barrel 40, it is done in the same manner as described above in connection with the embodiments shown in FIGS. 4 and 6. Accordingly, the needle assembly 70 contacts the retaining fingers 51 and pushes them into the open position. Once the needle assembly 70 is sufficiently inserted, the retaining fingers 51 move into the closed position and retain the hollow projection 74 of the needle assembly 70. If the needle assembly 70 is inserted through the aperture 48, it may be inserted in the same manner as, and at the same time as, the seal 53. If inserted through the aperture 48, the retaining fingers 51 need not move to the open position to accept the needle assembly 70.

In the embodiments shown in FIGS. 3-6, the cap member 10 may be mated with the closed end 44 of the syringe barrel 40 either before or after insertion of the needle assembly 70. In the embodiments shown in FIG. 7, the cap member 10 may be mated with the closed end 44 of the syringe barrel 40 after insertion of the needle seal 53.

The plunger assembly 100 is assembled by assembling the plunger frame 100, which already has the thumb pad 104 and sealing member 150 positioned thereon, and the retraction assembly 160. As explained above, the elastic member 164 is preferably molded directly to the mandrel 170 and catch member 190 105, to form the retraction assembly 160. If not formed integrally, the elastic member 164 is secured to the mandrel and catch member barbed shafts 180 and 194.

With the retraction assembly 160 complete, the mandrel 170 is inserted through the hollow shaft 140 passing through the plunger frame sealing end 130. In the embodiment shown in FIGS. 14 and 18, the mandrel 170 is inserted until the mandrel retaining ring 178 is secured by the sealing end internal annular ring 142. The mandrel retaining ring 178 forms a fluid tight seal with the plunger sealing end 130 proximate the pressure cone 138, thereby sealing the hollow shaft 140. The resilient stop 184 abuts against the rear surface of the sealing platform 132, thereby forming a fluid tight seal about that end of the hollow shaft 140. As the plunger assembly is withdrawn from the hollow body 42, a vacuum is created therein. The seal provided by the resilient stop 184 helps prevent air or other materials from be pulled past the mandrel 170 into the syringe body 42 by the internal vacuum force.

In the embodiment shown in FIGS. 15 and 19, the mandrel 170 is inserted to place the mandrel seal 171 within the first end 141 of the hollow shaft 140. The mandrel seal 171 may abut a pressure cone 138 or a lip (not shown) on the hollow shaft 140 that inhibits excessive insertion of the mandrel 170.

With the mandrel 170 in place, the plunger assembly 100 is ready to be inserted into the syringe barrel 40 through the open end 54. The annular seals 152 and 154 sealingly engage the inside of the syringe barrel 40 as the plunger assembly 100 is inserted. The plunger assembly 100 is inserted approximately half-way into the syringe barrel 40 until the catch member 190 105 abuts the shoulder 60, as shown in FIG. 21. The syringe 8 is ready for packaging and delivery. It should be noted that at this time the elastic member 164 is not tensioned. This helps increase the shelf life of the syringe 8 since the elastic member 164 is not under constant tension. If shelf life is not a concern, the catch member 190 105 can be secured to the retention assembly 120 prior to packaging, whereby the syringe 8 would have a preloaded elastic member.

After removing the syringe assembly 8 from the packaging, the operator can hold the syringe in a typical one hand manner, i.e. with two fingers abutting the grip member 56 and the thumb on the thumb pad 104. The operator presses on the thumb pad 104 to depress the plunger assembly 100 into the syringe barrel 40 with a substantially complete depression to expel air from the syringe hollow body 42. This is similar to standard syringe operation. As the plunger assembly 100 is depressed, the catch member 190 105 is retained by the shoulder 60 such that the catch member 190 105 cannot travel forward. However, the plunger frame 110 continues its forward travel. Since the catch member 190 105 is retained but the plunger frame 110 and secured mandrel 170 continue forward, the elastic member 164 begins to stretch and tension.

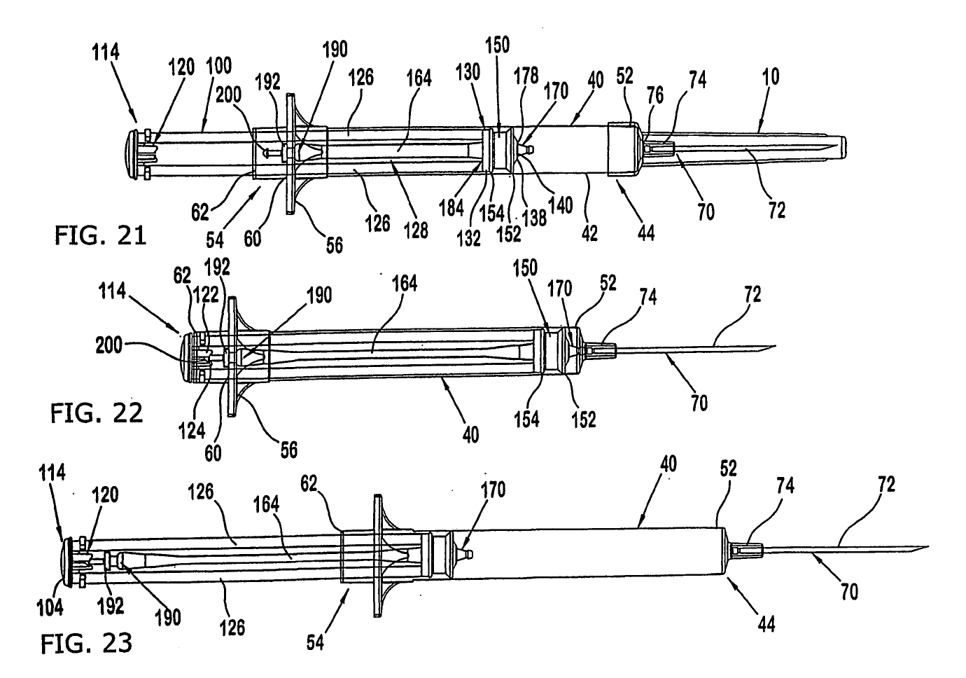

As travel continues forward, the catch element 200 109 of the catch member 190 105 is received by the retention assembly 120. In the embodiment shown in FIG. 20, the catch element is a geometrically configured catch tip 200. In the embodiment shown in FIGS. 12 and 13, the catch element is comprised of catch teeth 109 and the retention assembly is comprised of retention teeth 103. As shown in FIG. 22 for the embodiment with a catch tip 200, the catch element 200 109 is secured by the retention assembly 120 of the plunger frame 110. The elastic member 164 is thereby secured in a loaded condition between the secured mandrel 170 and the secured catch member 190 105. As the catch element 200 109 and retention assembly 120 mate, an audible “click” may occur to provide a signal of proper mating to the operator.

Additionally, the syringe barrel annular lip 62 adjacent the first end 114 of the plunger frame will discourage complete depression of the plunger assembly 100 as the catch element 200 109 is received by the retention assembly 120.

With the elastic member 164 loaded, the syringe 8 can be loaded in a typical fashion by removing the cap 10, inserting the needle 42 into a desired vial or the like, and withdrawing the plunger assembly 100 to draw up a desired dose as shown in FIG. 23. Since the elastic member 164 is tensioned between two components secured to the plunger frame 110, withdrawal of the plunger assembly 100 will not trigger the elastic member 164. Instead, the plunger assembly 100 will operate as a standard syringe plunger.

Once any air has been purged from the syringe barrel 40 in a known manner, the device 8 is ready for injection of the needle 72 into the patient. As stated above, the elastic member 164 is tensioned between two fixed components, and therefore, is not acting to move the plunger assembly 100 in either direction. As such, the user does not have to maintain constant pressure on the plunger assembly, but is free to hold the syringe 8 in the traditional dart like fashion between their thumb and forefinger of one hand, and use the other hand to pinch the patient's skin at the point of insertion for subcutaneous injection, spread the skin for intramuscular injection, and stabilize the skin for IV injection. These methods of injection are the generally preferred methods in the medical field.

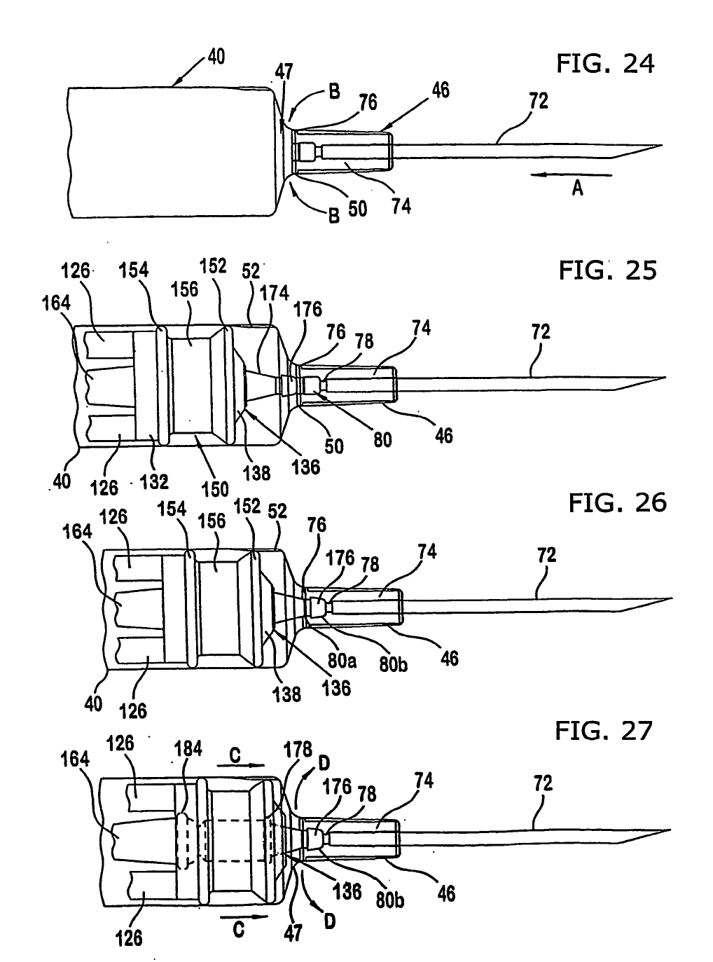

As the needle 72 is inserted, a rearward force, indicated by the arrow A in FIG. 24, is applied against the needle assembly 70. To resist this force, the needle assembly sealing ring 76 is secured within the retaining groove 50 in the embodiment shown in FIGS. 3 and 5. Additionally, since the syringe barrel tapered surface 47 about the truncated cone 46 is convex, the rearward force causes the syringe barrel surface to urge inward, as indicated by arrows B, thereby creating a tighter retention force about the needle assembly 70. Once the user has inserted the needle 72 into the patient, the user injects the substance into the patient by depressing the thumb pad 104. In the embodiments shown in FIGS. 4, 6, and 7, the force applied against the needle assembly is resisted by the retaining fingers 51.

In an embodiment shown in FIG. 25, upon substantial depression of the plunger assembly 100, the mandrel tip 176 begins to enter the needle assembly cavity 80. At approximately the same time, the first annular seal 152 meets and is deflected by the ramps 52 adjacent the closed end 44 of the syringe barrel 40, thereby breaking the fluid tight seal. Any fluid trapped between the plunger sealing member 150 and the needle assembly 70 is permitted to pass the deflected annular seal 152 into the open space around narrower portion 156. The second annular seal 154 may remain in sealing engagement with the syringe barrel 40 to prevent any unwanted inward or outward flow past the sealing member 150. However, the annular seal 154 may include a small passage to let trapped air about the narrower portion 156 escape.

In this embodiment, the mandrel tip 176 passes through the needle assembly cavity cylindrical portion 80a into the geometrically configured cavity hemispherical portion 80b whereby the mandrel 170 is secured to the needle assembly 70 as shown in FIG. 26.

At approximately the same time the mandrel 170 and needle assembly 70 attach, the retaining fingers 51 are moved into the open position by the plunger assembly 100 so that the needle assembly 70 may pass through the opening defined by the fingers 51. A holding force exerted by the fingers 51 on the needle assembly 70 is lower when the fingers 51 are in the open position than when the fingers 51 are in the closed position. The tension in the elastic member is sufficient to overcome any remaining holding force exerted by the fingers 51 on the needle assembly 70.

Once the mandrel 170 is secured to the needle assembly 70 as shown in FIG. 26, the mandrel tip 176 has moved as far into the needle assembly 70 as possible, yet the plunger frame 110 has not completed its full stroke. As such, continued force on the thumb pad 104 will continue to move the plunger frame 110 forward. Since the mandrel 170 position is fixed and the plunger frame 110 is being forced forward, the mandrel is pushed backward.

In the embodiment shown in FIGS. 14 and 18, the retaining ring 178 will be forced inward past the plunger sealing end retaining ring 136, thereby releasing the mandrel 170 as shown in FIG. 18. That is, the mandrel retaining ring 178 moves behind the retaining ring 136 as shown in phantom.

In the embodiments shown in FIGS. 15 and 19, as the needle user continues depressing the plunger, the plunger frame continues moving forward relative to the mandrel 170, so that the mandrel 170 is moved from a first position in the smaller-area first end 141 of the hollow shaft 140 to a second position in the larger-area second end 143 of the hollow shaft 140.

As the mandrel 170 moves from the first position toward the second position, the holding force between the hollow shaft 140 and the mandrel seal 171 is overcome and the mandrel seal 171 slides against the hollow shaft 140. The mandrel seal 171 moves with the mandrel 170 because the holding force between the mandrel seal 171 and the mandrel 170 is greater than the holding force between the mandrel seal 171 and the hollow shaft 140.

Once the mandrel seal 171 moves into the larger area of the second end 143 of the hollow shaft 140, the mandrel seal 171 either expands away from the mandrel 170 or remains engaged with the mandrel 170. The seal 171 would expand if it were compressed around the mandrel 170 by the hollow shaft 140 during the assembly of the syringe. If the seal 171 expands, the area defined by an opening in the seal 171 (the inner seal area) preferably becomes large enough as the seal 171 moves from the first position to the second position, such that the mandrel 170 may retract the needle through the inner seal area.

In any case, the mandrel seal 171 no longer secures the mandrel 170 to the hollow shaft 140 after the mandrel seal 171 moves from the first position to the second position within the hollow shaft 140.

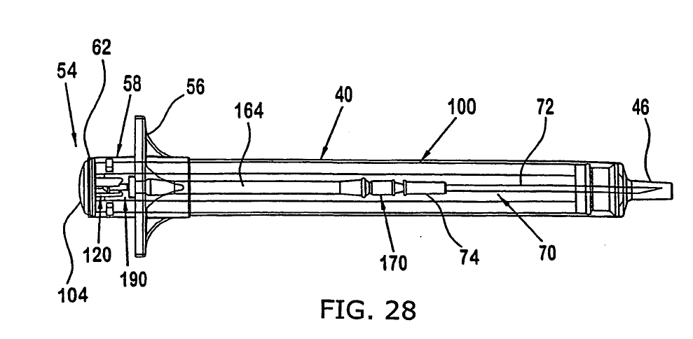

Since the mandrel 170 is under the load of the elastic member 164 but no longer secured to the hollow shaft 140, the load of the elastic member 164 automatically retracts the mandrel 170 into the plunger frame 110 between the connecting rods 126. Through the connection of mandrel tip 176 and the geometrically configured needle assembly cavity 80, the needle assembly 70 is also retracted into the plunger frame 110, as shown in FIG. 28.

In one embodiment, the tapered pressure cone 138 on the sealing end of the plunger frame 110 contacts the convex, tapered portion 47 of cone 46 and causes it to spread slightly. This reduces the retaining force of cone 46 on the needle assembly 70 to assist retraction of the needle assembly 70. Furthermore, since the tapered portion 47 is convex, the forward fluid and plunger force, as indicated by arrow C in FIG. 27, urge the tapered portion 47 outward, as indicated by arrows D, further easing the retaining force and thereby reducing the requisite retraction force. This flexing preferably occurs simultaneously or slightly after the mandrel 176 enters the hemispherical portion 80a of the needle assembly cavity 80.

Referring again to FIG. 28, as the plunger assembly 100 completes its stroke, the thumb pad 104 enters the open cavity 58 at the end of the syringe barrel 40. In the preferred embodiment, the thumb pad 104 is manufactured from a resilient material which sealingly engages the syringe barrel 40 wall, thereby closing the open end 54 and preventing any inadvertent fluid flow out of the syringe barrel 40. Additionally, since the thumb pad 104 enters and is recessed in the open cavity 58, it makes it difficult for anyone to inadvertently or intentionally remove the plunger assembly 100 and expose the used needle 72. The thumb pad 104 preferably has a semi-domed configuration which enhances its inaccessibility. Additionally, the thumb pad 104 is preferably inserted past the inner annular lip 62 and retained thereby, further enhancing inaccessibility.

B. Fluid Retention Systems

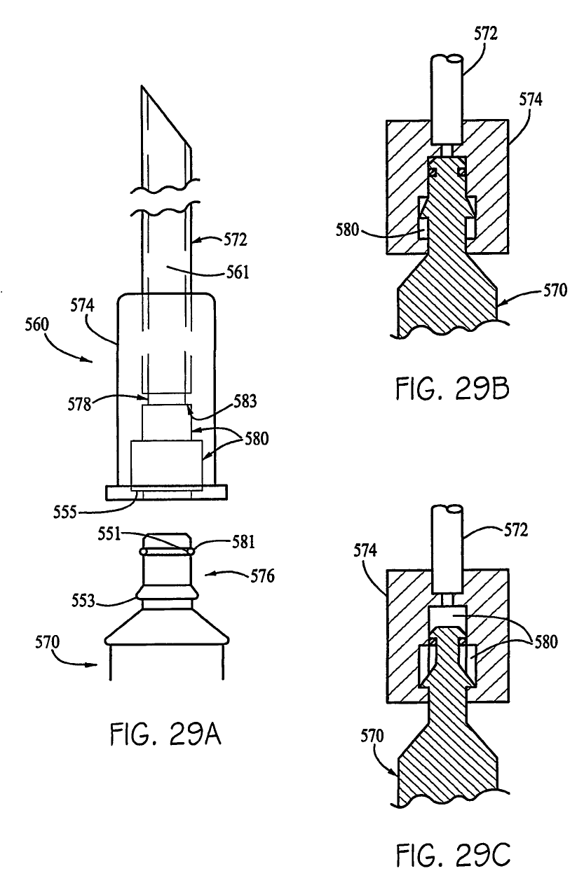

FIGS. 29A, 29B, and 29C depict an embodiment of a fluid retention system that includes a cylinder 580 and a piston 576. The piston 576 fits within and slidingly engages the cylinder 580. The cylinder 580 may fluidly communicate with the needle passage 561 of the needle 572 through an interior passage 578 of the needle assembly 560. In this embodiment, the piston 576 is integral with the mandrel 570 and the cylinder 580 is defined within the hollow projection 574 of the needle assembly 560 of a retractable syringe.

After the syringe is charged with fluid to be injected into, a patient, the plunger assembly may be depressed to inject the patient. When the plunger assembly is depressed substantially completely, the piston 576 begins to enter the cylinder 580. As the plunger is further depressed, a shoulder 553 of the piston 576 slides past a lip 555 of the cylinder 580 and enters the cylinder 580, the abutment of the shoulder 553 and lip 555 securing the piston inside the cylinder. The cylinder preferably is defined at least partially by a resilient material such that the shoulder 553 may urge the lip 555 open as it moves into the cylinder 580, and the lip 555 may return to a relaxed state after the shoulder 553 moves past the lip. As the plunger assembly is depressed still further, the piston 576 contacts a stop 583 that inhibits further forward movement of the piston 576 relative to the cylinder 580.

As the plunger is further depressed, the mandrel 570 becomes disengaged from the hollow shaft that initially secures the mandrel 570 in place as described in connection with FIGS. 15 and 19, and the elastic member pulls the mandrel 570 into the hollow body.

As the mandrel retracts into the barrel, the needle assembly 560 initially tends not to retract with the mandrel because of inertia of the needle assembly or because of holding forces exerted on the needle assembly by the patient's skin or by parts of the needle assembly that contact the rest of the syringe, such as the sealing ring and retaining groove described in connection with FIGS. 3 and 5. The piston 576 initially moves relative to the cylinder 580, increasing the volume of a vacuum cavity located between the piston 576 and the tip of the needle 572. As the volume of the cavity increases, the pressure inside the cavity initially decreases and remains at a decreased level until air or other fluid entering the cavity equalizes the pressure in the cavity with ambient pressure.

The pressure drop inside the vacuum cavity preferably tends to draw into the cavity any fluid remaining in the needle passage 561, such as fluid remaining at the tip of the needle 572, thereby inhibiting fluid loss from the needle 572 during retraction. Preferably, the vacuum cavity is substantially sealed other than at the needle tip and at least partially open at the needle tip so that air or other fluid may enter the vacuum cavity only (or at least most easily) through the needle tip and is drawn away from the needle tip into the vacuum cavity. To provide the preferred sealing, the piston 576 substantially sealingly engages the cylinder 580. This sealing may be accomplished with a piston seal 581, such as an annular ring, that sealingly engages the piston 576 and the cylinder 580. The piston 576 may have a seal retaining groove 551 that positions and retains the piston seal 581.

After the piston 576 retracts and moves relative to cylinder 580 sufficiently, the shoulder 553 of the piston 576 abuts the lip 555 of the cylinder 580, inhibiting further relative movement of the piston 576 to the cylinder 580. As the piston 576 further retracts, it retracts the needle assembly 560 along with it, retracting the needle 572 into the hollow barrel of the syringe.

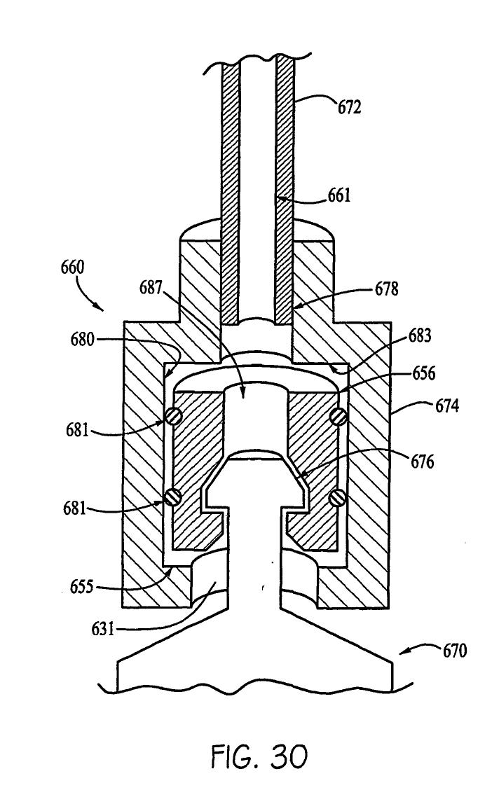

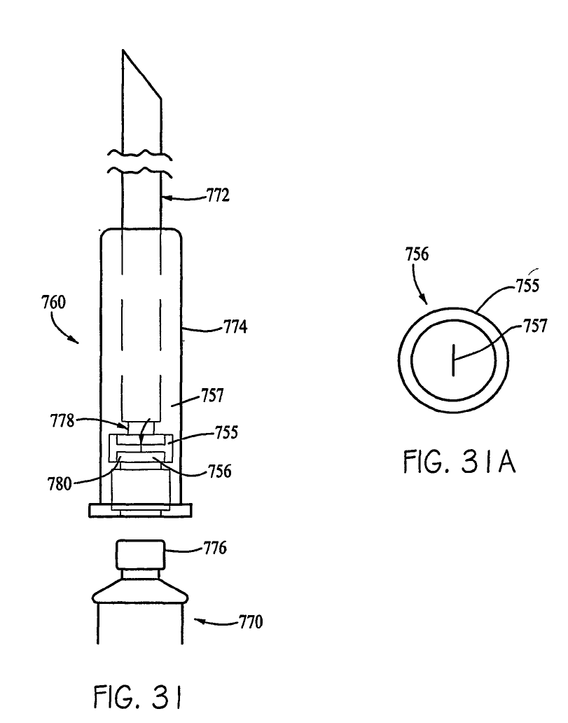

FIG. 30 depicts another embodiment of a fluid retention system that includes a cylinder 680 and a piston 656. In this embodiment, the piston 656 is secured in and slideable within the cylinder 680. The piston 656 is secured from excessive axial movement within the cylinder 680 by a lip 655 and a stop 683 disposed opposite each other. The piston has a piston passage 687 that is in fluid communication with the needle passage 661 through an interior passage 678 of the needle assembly 660.

The mandrel 670 has a geometrically configured tip 656 and the piston passage 687 is configured to engage the geometrically configured tip 676 of the mandrel 670. The hollow projection 674 defines a keyway 631 through which the geometrically configured tip 676 of the mandrel 670 may enter to engage the piston passage 687. In this embodiment, the cylinder 680 is defined within the hollow projection 674 of the needle assembly 660 of a retractable syringe.

After the syringe is charged with fluid to be injected into a patient, the plunger assembly may be depressed to inject the patient. When the plunger assembly is depressed substantially completely, the mandrel tip 676 enters the keyway 631 and pushes against the piston. As the plunger assembly is further depressed, the mandrel tip 676 engages the piston passage 687 and the mandrel tip 676 moves the piston 656 toward the needle 672 until the piston 656 contacts the top 683 of the cylinder 680, inhibiting further forward movement of the piston 656 relative to the cylinder 680.

As the plunger is further depressed, the mandrel 670 becomes disengaged from the hollow shaft that initially secures the mandrel 670 in place as described in connection with FIGS. 15 and 19, and the elastic member pulls the mandrel 670 into the hollow body of the syringe.

As the mandrel 670 retracts into the barrel, the needle assembly 660 initially tends not to retract with the mandrel 670 because of inertia of the needle assembly 660 or because of holding forces exerted on the needle assembly 660 by the patient's skin or by parts of the needle assembly that contact the rest of the syringe, such as the sealing ring and retaining groove described in connection with FIGS. 3 and 5. The piston 656 initially moves relative to the cylinder 5=680, increasing the volume of a vacuum cavity located between the piston 656 and the tip of the needle 672. As the volume of the cavity increases, the pressure inside the cavity initially decreases and remains at a decreased level until air or other fluid entering the cavity equalizes the pressure in the cavity with ambient pressure.

The pressure drop inside the vacuum cavity preferably tends to draw into the cavity any fluid remaining in the needle passage 561, such as fluid remaining at the tip of the needle 572, thereby inhibiting fluid loss from the needle 572 during retraction. Preferably, the vacuum cavity is substantially sealed other than at the needle tip and at least partially open at the needle tip so that air or other fluid may enter the vacuum cavity only (or at least most easily) through the needle tip and is drawn away from the needle tip into the vacuum cavity. To provide the preferred sealing, the mandrel tip 676 preferably substantially sealingly engages the piston passage 687 and the piston 656 substantially sealingly engages the cylinder 680. Sealing may be aided by one or more piston seals 681, which may comprise an annular ring. The piston 656 may have a seal retaining groove (not shown) that positions and retains the piston seal 681.

After the piston 576 retracts and moves relative to cylinder 580 sufficiently, the 553 of the piston 656 abuts the lip 655 of the cylinder 680, inhibiting further relative movement of the piston 656 to the cylinder 680. As the piston 656 further retracts, it retracts the needle assembly 660 along with it, retracting the needle 672 into the hollow barrel of the syringe.

FIGS. 31 and 31A depict another embodiment of a fluid retention system. This embodiment includes a diaphragm cavity 780 and a diaphragm 756 having a centrally-disposed slit 757 and a spacer 755. The diaphragm 756 is disposed within and preferably sealingly engages a diaphragm cavity 780 defined by the hollow projection 774. The slit cavity 780 may fluidly communicate with the needle passage 761 through an interior passage 778 of the needle assembly 760.

This embodiment is particularly well-suited for use in either retractable syringes or conventional, non-retractable syringes. In a retractable syringe, the diaphragm preferably is disposed in the hollow projection 774 or otherwise formed into the needle assembly 760, but alternately may be disposed outside of the needle assembly 760 near the closed end of the hollow body and connected with the needle assembly 760. Further, the diaphragm may include a geometric configuration securable with the mandrel 770.

The diaphragm selectively provides fluid communication through the slit. The diaphragm preferably comprises a resilient material that bulges under fluid pressure and relaxes when the pressure is decreased. Such material may be, for example, a polymer or a flexible membrane. When the diaphragm is relaxed and not bulging, fluid communication through the slit is inhibited, and when the diaphragm is bulging under fluid pressure that is not equal on both sides of the diaphragm, fluid communication through the slit is provided. The spacer 755 provides clearance within the hollow projection 774 to accommodate bulging of the diaphragm 756.

As the plunger assembly is withdrawn to charge the syringe or depressed to inject the charge into a patient, pressure in the hollow barrel of the syringe increases. The fluid pressure acts against the diaphragm 756, urging the slit 767 and allowing fluid communication through the diaphragm 756. When the plunger assembly is depressed substantially completely, a geometrically configured tip 776 of the mandrel 770 enters and engages the needle assembly 760 and further forward movement of the tip 776 relative to the needle assembly 760 is inhibited, allowing the fluid pressure on the diaphragm to drop.

When fluid pressure on the diaphragm drops, the bulging in the flexible diaphragm subsides and fluid no longer flows through the slit. As the bulge subsides, the volume of a vacuum cavity located between the diaphragm and the needle tip increases. As the volume of the cavity increases, the pressure inside the cavity initially decreases and remains at a decreased level until air or other fluid entering the cavity equalizes the pressure in the cavity with ambient pressure. The diaphragm substantially sealingly engages the slit cavity and is substantially sealed other than at the needle tip. As explained in connection with other embodiments, the pressure drop inside the cavity preferably tends to draw into the vacuum cavity any fluid remaining in the needle passage, thereby inhibiting fluid loss from the needle during retraction of the needle.

As the plunger is further depressed, the mandrel 770 becomes disengaged from the hollow shaft that initially secures the mandrel 770 in place as described in connection with FIGS. 15 and 19, and the elastic member pulls the mandrel 770 into the hollow body.

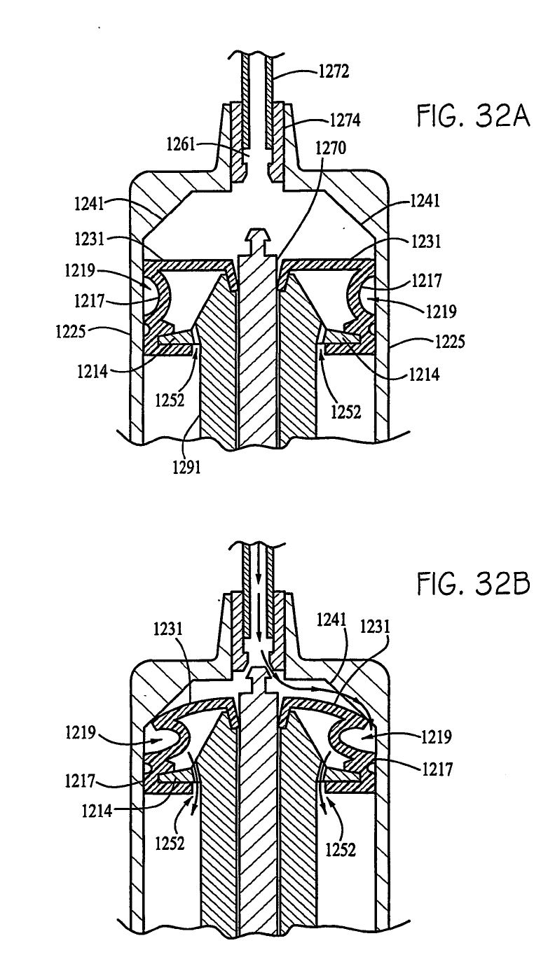

FIGS. 32A and 32B depict another embodiment of a fluid retention system that includes a plunger seal 1217 secured with the sealing platform 1214 of the plunger assembly and sealingly engaging the plunger assembly and the hollow body 1225 of the syringe. The plunger seal 1217 and the hollow body 1225 together define an annular cavity 1219. Ribs 1241 are positioned near the closed end of the hollow body 1225. Preferably, there are three or four ribs positioned radially about the closed end of the hollow body 1225.

After the syringe is charged with fluid to be injected into a patient, the plunger assembly may be depressed to inject the patient. As the plunger assembly is depressed, the seal is moved toward the closed end of the hollow body. When the plunger is substantially completely depressed, the top 1231 of the seal contacts one or more ribs 1241 and is prevented from moving further forward. As the plunger is further depressed, it moves the bottom portion of the seal toward the top 1231 of the seal, compressing the seal. As the seal is compressed, the top surface of the seal no longer sealingly engages the hollow body, such that the annular cavity fluidly communicates with the needle passage 1261. This fluid communication preferably is provided through spaces between the ribs. Alternately, this communication may be provided through a passage in the seal (not shown). The bottom portion of the plunger seal continues to sealingly engage the plunger assembly with the barrel even while the seal is being compressed.

As the seal is compressed, the annular cavity expands into the seal, increasing the volume of a vacuum cavity located between the annular cavity and the needle tip, and the mandrel 1270 engages the hollow projection 1274 of the needle assembly.

Preferably, the vacuum cavity is substantially sealed other than at the needle tip. As the annular cavity expands and the cavity volume increases, a pressure drop is created in the vacuum cavity, tending to draw into the cavity any fluid remaining at the tip of the needle. As the plunger is further depressed, the mandrel 1270 becomes disengaged from the hollow shaft that initially secures the mandrel 1270 in place as described in connection with FIGS. 15 and 19, and the elastic member pulls the mandrel 1270 into the hollow body 1225 of the syringe.

The annular cavity may expand after the mandrel engages the needle assembly. In such a case, fluid communication between the annular cavity and the needle passage 1261 may be provided by a gap (not shown) between the mandrel and the interior surface of the hollow projection 1274. Such a gap may be in the form of a groove in the interior surface of the hollow projection 1274 or a groove in the mandrel.

As the annular cavity expands into the internal cavity of the plunger seal, air preferably is displaced from the internal cavity of the plunger seal through one or more air vents 1252 that provide fluid communication with the interior portion of the hollow body located proximally of the seal. If the air is not displaced from the cavity, then pressure in the internal cavity will increase as the annular cavity expands. This increasing pressure may inhibit the expansion of the annular cavity.

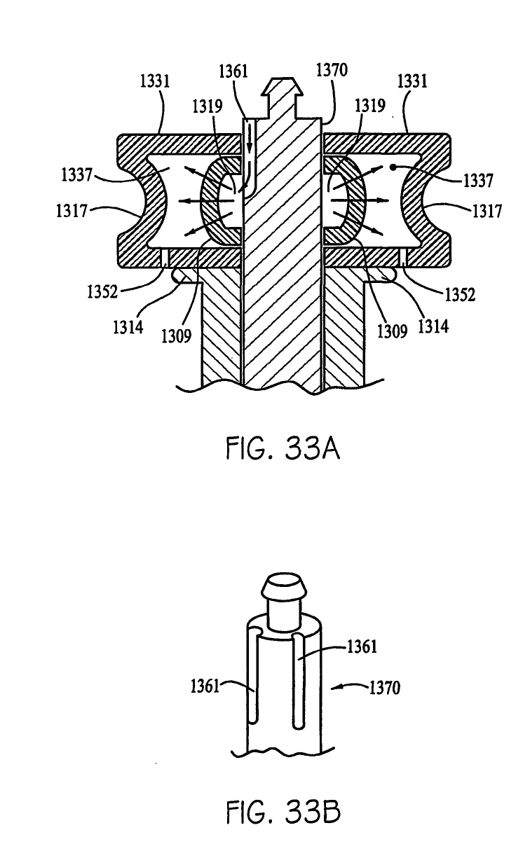

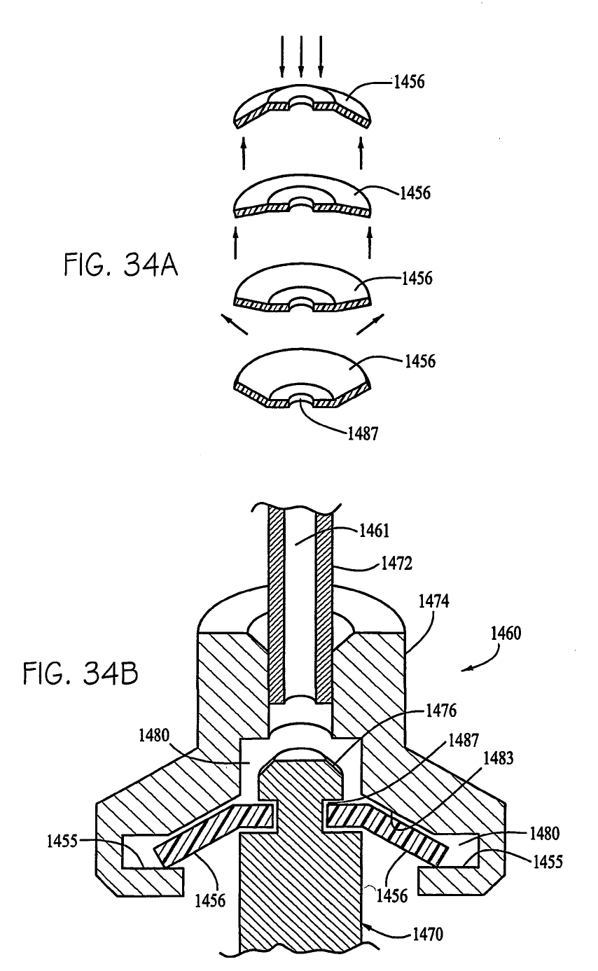

FIGS. 33A and 33B depict another embodiment of a fluid retention system that includes a plunger seal 1317 secured with the sealing platform 1314 of the plunger assembly and sealingly engaging the plunger assembly and the hollow body of the syringe. The plunger seal has an internal collar that is positioned in an internal cavity 1337 of the plunger seal. The internal collar defines a central cavity 1319 together with the mandrel 1370. The central cavity fluidly communicates with the needle passage. Fluid communication is preferably provided by one or more grooves 1361 in the mandrel, but alternately may be provided by a passage through the top portion 1331 of the plunger seal.

After the syringe is charged with fluid to be injected into a patient, the plunger assembly may be depressed to inject the patient. As the plunger assembly is depressed, the seal is moved toward the closed end of the hollow body. When the plunger is substantially completely depressed, the top portion 1331 of the seal contacts the closed end of the hollow body of the syringe and is prevented from moving further forward. As the plunger is further depressed, it moves the bottom portion of the seal toward the top portion 1331 of the seal, compressing the seal and the internal collar. As the internal collar is compressed, the volume of the central cavity increases, increasing the volume of a vacuum cavity located between the internal collar and the needle tip, and the mandrel engages the needle assembly.

Preferably, the vacuum cavity is substantially sealed other than at the needle tip. As the central cavity expands and the cavity volume increases, a pressure drop is created in the vacuum cavity, tending to draw into the cavity any fluid remaining at the tip of the needle. As the plunger is further depressed, the mandrel 1370 becomes disengaged from the hollow shaft that initially secures the mandrel 1370 in place as described in connection with FIGS. 15 and 19, and the elastic member pulls the mandrel 1370 into the hollow body of the syringe.

The central cavity may expand after the mandrel engages the needle assembly. In such a case, fluid communication between the central cavity and the needle passage may be provided by a gap (not shown) between the mandrel and the interior surface of the hollow projection. Such a gap may be in the form of a groove in the interior surface of the hollow projection 1274 or a groove in the mandrel.

As the internal collar expands into the internal cavity of the plunger seal, air preferably is displaced from the internal cavity of the plunger seal through one or more air vents 1352 that provide fluid communication with the interior portion of the hollow body located proximally of the seal. If the air is not displaced from the cavity, then pressure in the internal cavity will increase as the collar expands. This increasing pressure may inhibit the expansion of the collar.

FIGS. 34A and 34B depict an embodiment of a fluid retention system that includes a washer cavity 1480 and a washer 1456 contained by and moveable within the washer cavity. The washer cavity 1480 is defined within the hollow projection 1474 of the needle assembly 1460. The washer is invertable between and stable in each of a first configuration and a second configuration, both shown in FIG. 34A. The washer substantially sealingly engages the washer cylinder as the washer is inverted (i.e., flipped inside-out) between the first configuration and the second configuration. The washer 1456 has a washer passage 1487 providing fluid communication through the washer, and having a geometric configuration securable with the mandrel. The washer cavity 1480 fluidly communicates with the needle passage 1461 of the needle 1472.

The washer of this embodiment is contained in the washer cavity, but alternately may be connected with or integral with the mandrel and enter the washer cavity when the plunger assembly is depressed substantially completely.

After the syringe is charged with fluid to be injected into a patient, the plunger assembly may be depressed to inject the patient. When the plunger assembly is depressed substantially completely, the geometrically configured tip 1476 of the mandrel enters and engages the washer passage, and moves the washer toward an upper surface 1483 of the washer retainer that inhibits further forward movement of the washer 1456 relative to the washer cavity 1480.

As the plunger is further depressed, the mandrel 1470 becomes disengaged from the hollow shaft that initially secures the mandrel 1470 in place as described in connection with FIGS. 15 and 19, and the elastic member pulls the mandrel 1470 into the hollow body.

As the mandrel retracts into the barrel, the needle assembly 1460 initially tends not to retract with the mandrel because of inertia of the needle assembly or because of holding forces exerted on the needle assembly by the patient's skin or by parts of the needle assembly that contact the rest of the syringe, such as the sealing ring and retaining groove described in connection with FIGS. 3 and 5. As the mandrel retracts, the washer is pulled away from the needle and comes into contact with a lip 1455 of the washer cavity 1480. The lip constrains the washer about its circumference, and the retracting mandrel influences the center portion of the washer to move relative to the circumferential portion. The mandrel applies sufficient force to cause the washer to invert. The washer being unstable until it reaches its second stable configuration, the washer while under the influence of the retracting mandrel tends to move toward the second configuration until it is reached. When the washer inverts, a volume of a vacuum cavity located between the washer and the tip of the needle increases, decreasing the pressure inside the cavity.

The pressure drop inside the vacuum cavity preferably tends to draw into the cavity any fluid remaining in the needle passage 561, such as fluid remaining at the tip of the needle 572, thereby inhibiting fluid loss from the needle 572 during retraction. Preferably, the vacuum cavity is substantially sealed other than at the needle tip and at least partially open at the needle tip so that air or other fluid may enter the vacuum cavity only (or at least most easily) through the needle tip and is drawn away from the needle tip into the vacuum cavity. To provide the preferred sealing, the geometrically configured tip of the mandrel preferably substantially sealingly engages the washer passage, and the washer preferably sealingly engages the washer cavity. This sealing may be accomplished with a washer seal (not shown), such as an annular ring, that sealingly engages the washer 1476 and the washer cavity 1480.

As the mandrel further retracts, it retracts the needle assembly 1460 along with it, retracting the needle 1472 into the hollow barrel of the syringe.

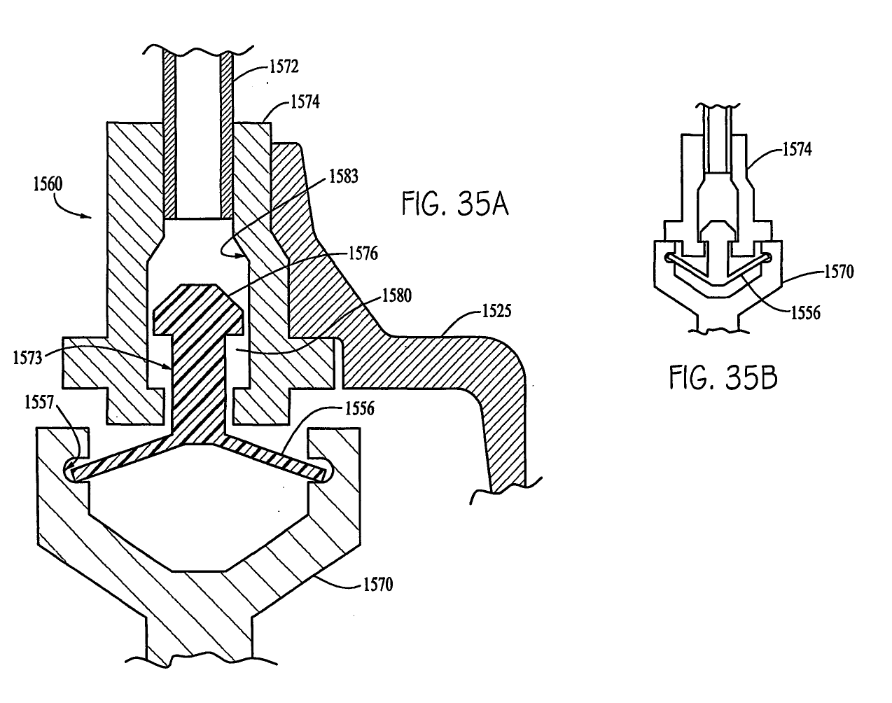

FIGS. 35A and 35B depict an embodiment of a fluid retention system that includes an anvil cylinder 1580 and an anvil 1573 slideably secureable with the anvil cylinder. The anvil cylinder 1580 is defined within the hollow projection 1574 of the needle assembly 1560 and in fluid communication with the needle passage. The anvil 1573 has an anvil base 1556 and an anvil head 1576 having a geometric configuration that is secureable with the anvil cavity 1580 of the needle assembly. The anvil base is invertable between and stable in each of a first configuration shown in FIG. 35A and a second configuration shown in FIG. 35B. The anvil head substantially sealingly engages the anvil cylinder as the anvil base is inverted (i.e., flipped inside-out) between the first configuration and the second configuration. An anvil base cavity is defined by the retraction member and includes a retaining groove disposed about a perimeter of the cavity. The anvil base preferably is seated in the retaining groove.

After the syringe is charged with fluid to be injected into a patient, the plunger assembly may be depressed to inject the patient. When the plunger assembly is depressed substantially completely, the geometrically configured anvil head 1576 enters and slidingly engages the anvil cylinder, and moves forward toward the needle until it contacts a stop 1583 in the cylinder that inhibits further forward movement of the anvil head 1576 relative to the anvil cylinder 1580, or until the anvil base contacts the hollow projection.

As the plunger is further depressed, the anvil base inverts to its second configuration and the mandrel 1570 becomes disengaged from the hollow shaft that initially secures the mandrel 1570 in place as described in connection with FIGS. 15 and 19, and the elastic member pulls the mandrel 1570 into the hollow body.

As the mandrel retracts and the anvil inverts, the anvil head is pulled away from the needle. The needle assembly 1560 initially tends not to retract with the mandrel because of inertia of the needle assembly or because of holding forces exerted on the needle assembly by the patient's skin or by parts of the needle assembly that contact the rest of the syringe, such as the sealing ring and retaining groove described in connection with FIGS. 3 and 5. The anvil moves relative to the anvil cylinder away from the needle. When the anvil base inverts and the anvil head slides relative to the anvil cylinder, a volume of a vacuum cavity located between the anvil head and the tip of the needle increases, decreasing the pressure inside the vacuum cavity.

The pressure drop inside the vacuum cavity preferably tends to draw into the cavity any fluid remaining in the needle passage 1561, such as fluid remaining at the tip of the needle 1572, thereby inhibiting fluid loss from the needle 1572 during retraction. Preferably, the vacuum cavity is substantially sealed other than at the needle tip and at least partially open at the needle tip so that air or other fluid may enter the vacuum cavity only (or at least most easily) through the needle tip and is drawn away from the needle tip into the vacuum cavity. To provide the preferred sealing, the geometrically configured anvil head preferably substantially sealingly engages the anvil cylinder. As the mandrel further retracts, it retracts the needle assembly 1560 along with it, retracting the needle 1452 into the hollow barrel of the syringe.

It should be appreciated that many of the embodiments described herein are adaptable for use in non-retractable syringes. Embodiments that rely on the retraction mechanism to move a component of the vacuum assembly, such as a piston or stretchable tube may be implemented in a non-retractable syringe by using a biasing element to substitute for the retraction mechanism. For example, a spring may be interposed between the cylinder and the piston of the embodiment described in FIG. 30? so that, after the piston is pressed toward the top of the cylinder during injection of a patient, the piston driver drives the piston back down toward the bottom of the cylinder, creating a pressure drop and tending to draw fluid away from the needle tip.

What is claimed is:

1. A retractable syringe comprising:
a hollow body having an open proximal end and a distal end defining an aperture;
a needle assembly selectively secureable with the hollow body and passing through the aperture, the needle assembly having an internal passage, a hollow projection and a needle positioned in the hollow projection, the needle having a needle passage and a needle tip;
a plunger assembly slidably positioned in the hollow body and having a distally disposed sealing platform and a retraction member including a mandrel, releasably secured to the sealing platform and engageable with the needle assembly; and
a fluid retention system between the sealing platform and the needle, the fluid retention system including a variable volume cavity in fluid communication with the needle passage and otherwise sealed, with the mandrel secured to the sealing platform and expandable without retraction of the needle assembly.

2. The syringe of claim 1, the fluid retention system further including a cylinder fixed to one of the mandrel and the needle assembly and a piston fixed to the other of the mandrel and the needle assembly and slidable within and substantially sealingly engageable with the cylinder with the plunger assembly at the distal end of the hollow body.

3. The syringe of claim 2, the cylinder being on the proximal end of the needle assembly and the piston being on the distal end of the mandrel, the piston being securable with the cylinder.

4. The syringe of claim 3, the piston including a shoulder and the cylinder including an inwardly extending lip, the shoulder abutting the lip to secure the piston with the cylinder.

5. The syringe of claim 2, the cylinder being on the proximal end of the needle assembly and the piston being slideably secured in the cylinder and having a piston passage with a geometric configuration, the piston passage providing fluid communication through the piston, the mandrel having a geometrically configured tip engageable with the geometric configuration of the piston passage, and the needle assembly having a keyway through which the geometrically configured tip of the mandrel may pass.

6. The syringe of claim 1, the fluid retention system further including a flexible diaphragm extending across the internal passage and having a centrally disposed slit selectively providing fluid communication through the diaphragm, the slit member bulging under the influence of a pressure imbalance in either direction to allow fluid flow through the slit, and the slit member relaxing in the absence of a fluid pressure imbalance and inhibiting fluid flow through the slit.

7. The syringe of claim 6, the internal passage having a geometric configuration securable with the mandrel.

8. The syringe of claim 1, the fluid retention system further including a plunger seal connected with the sealing platform of the plunger assembly, the plunger seal with the hollow body defining an annular cavity, a volume of the annular cavity increasing and a top portion of the plunger seal releasing from the hollow body when the plunger seal is compressed toward the sealing platform and at least one ramp member connected with the hollow body proximate the distal end of the hollow body, the at least one ramp member abutting the plunger seal and facilitating compression of the plunger seal and release of sealing engagement between the plunger seal and the hollow body with the plunger assembly approaching the distal end of the hollow body.

9. The syringe of claim 1, the fluid retention system further including a plunger seal connected with the sealing platform of the plunger assembly, the plunger seal having an internal collar defining with the retraction member a collar cavity fluidly communicating with the needle passage, a volume of the collar cavity increasing when the plunger seal is compressed.

10. The syringe of claim 9, the plunger seal abutting the hollow body proximate the distal end of the hollow body when the plunger is substantially completely depressed.

11. The syringe of claim 9, the retraction member having a groove providing fluid communication between the collar cavity and the needle passage prior to engagement of the retraction member with the needle assembly.

12. The syringe of claim 1, the fluid retention system further including a washer cavity defined by the needle assembly and in fluid communication with the needle passage, and a washer contained by and moveable within the washer cavity and having a washer passage providing fluid communication through the washer, the washer passage having a geometric configuration securable with the mandrel, the washer being invertable between and stable in each of a first configuration and a second configuration, the washer substantially sealingly engaging the washer cylinder as the washer is inverted between the first configuration and the second configuration.

13. The syringe of claim 1, the fluid retention system further including an anvil cylinder defined by the needle assembly and in fluid communication with the needle passage, an anvil base cavity defined by the retraction member and having a retaining groove disposed about a perimeter of the cavity, and an anvil having an anvil head with a geometric configuration securable with the needle assembly, slidable within the anvil cylinder, and substantially sealingly engageable with the anvil cylinder, the anvil having an anvil base seated in the retaining groove and invertable between and stable in each of a first configuration and a second configuration.

14. A method of inhibiting fluid loss following injection from a syringe having a body, a plunger assembly slidably positioned in the body and a needle assembly with a needle having a needle passage and selectively engaged with the body, comprising
reducing pressure in the needle passage after the plunger assembly has been substantially completely depressed;
retracting the needle assembly into the body of the syringe following reduction of pressure in the needle passage.

15. The method of claim 14, reducing pressure including expanding a volume between the plunger assembly and the needle assembly, the volume being open to the needle passage.
